(19) United States
(12) Patent Application Publication
(10) Pub. No.: US 2011/0002846 A1
(43) Pub. Date: Jan. 6, 2011

(54) CD24 AS A BRAIN TUMOR STEM CELL MARKER AND A DIAGNOSTIC AND THERAPEUTIC TARGET IN PRIMARY NEURAL AND GLIAL TUMORS OF THE BRAIN

(75) Inventors: Steven A. Goldman, Webster, NY (US); Fraser Sim, Batavia, NY (US); Romane Melanie Auvergne, Rochester, NY (US)

Correspondence Address: NIXON PEABODY LLP - PATENT GROUP, 1100 CLINTON SQUARE, ROCHESTER, NY, 14604, US

(73) Assignee: Univeristy of Rochester, Rochester, NY (US)

(21) Appl. No.: 12/809,491

(22) PCT Filed: Dec. 22, 2008
(86) PCT No.: PCT/US2008/087977

§ 371 Date: Sep. 16, 2010

Related U.S. Application Data
(60) Provisional application No. 61/016,381, filed on Dec. 21, 2007.

(51) Int. Cl. A61K 51/10; A61K 39/395; G01N 33/53; C12Q 1/68; A61K 49/00; A61P 35/00

(52) U.S. Cl. 424/1.49; 424/134.1; 436/86; 436/94; 436/501; 435/6; 435/7.21; 424/9.1

(57) ABSTRACT

The present invention is directed to methods of treating a primary brain tumor and preventing the migratory spread of a primary brain tumor in a subject. These methods involve utilizing the CD24 surface protein selectively expressed on tumor progenitor cells as a therapeutic target as well as a means for directing oncolytic therapeutics directly to the tumor site. The present invention further relates to methods of diagnosing the presence of a brain tumor and monitoring the status of the brain tumor in a subject based on CD24 expression in tumor progenitor cells.

This application claims the benefit of U.S. Provisional Patent Application Ser. No. 61/016,381 filed Dec. 21, 2007.

### FIELD OF THE INVENTION

The present invention relates to methods of treating a primary brain tumor, preventing its migratory spread, and diagnosing a primary brain tumor.

### BACKGROUND OF THE INVENTION

In a variety of both hematopoietic and solid tissue tumors, it has been shown that cancers may arise from neoplastic tissue-specific stem cells, that may remain distinct and separable from their neoplastic daughter cells. The cancer stem cell hypothesis thereby argues that cancers are sustained by expansion from relatively small numbers of neoplastic progenitors, that are required for tumor expansion. These neoplastic progenitors may be responsible not only for initiating brain cancers, but for regenerating them after cytotoxic treatment, leading ultimately to clinical relapse and treatment failure.

Defining those genes and signaling pathways that distinguish glial tumor stem cells from the native progenitor cells from which they derive is thus of critical importance to the development of antineoplastics targeted against malignant glioma. The different phenotypes of human brain tumors may comprise a hierarchy of neoplastic progenitor cells, with distinct tumors corresponding to the transformed derivatives of cells transformed at distinct points in their lineage progression. As a result, the daughter cells of transformed progenitor cells may be sufficiently distinct from their parents in both their expansion and growth control, that therapy directed at their derivative tumors may be neither appropriate nor effective against the parental transformed progenitor clone. Of note, this differs somewhat from the cancer stem cell hypothesis, which would argue that abolition of the parental progenitor, or tumor stem cell, would be sufficient to destroy a given tumor, whose derivative phenotypes would be incapable of self-renewal. Rather, we suggest that derivative phenotypes may well be capable of autonomous self-renewal once generated, but that cytotoxic therapy directed against them, without abolition of their underlying parental transformant, is destined for relapse and ultimate treatment failure. The therapeutic implication is that oncolytic therapy may need to be directed not solely at either the tumor stem cell or its derivatives, but rather at both, through fundamentally different mechanistic strategies.

The present invention is directed to overcoming these and other deficiencies in the art.

### SUMMARY OF THE INVENTION

A first aspect of the present invention relates to a method of treating a primary brain tumor in a subject. This method involves administering to the subject a CD24-specific targeting component under conditions effective to treat the primary brain tumor in the subject.

Another aspect of the invention relates to a method of preventing the migratory spread of a primary brain tumor in a subject. This method involves administering to the subject a CD24-specific targeting component under conditions effective to prevent the migratory spread of the primary brain tumor within the subject.

A third aspect of the present invention relates to a method of diagnosing the existence of a brain tumor in a subject. This method involves providing a patient sample and analyzing the patient sample for the presence of a CD24 antigen or a CD24-encoding nucleic acid molecule, thereby indicating the potential presence of a brain tumor in the subject.

Another aspect of the present invention relates to a method of diagnosing the existence of or monitoring the status of a brain tumor in a subject. This method involves providing a subject and analyzing the subject for the presence or anatomical distribution of a brain tumor based on the presence of a CD24 antigen thereby diagnosing the existence of or monitoring the status of a brain tumor in the subject.

As a selective marker of brain tumor-initiating cells within primary glioma and other primary brain tumors, the significant expression of which is not shared by non-tumor brain cells, CD24 represents a selective antigen with which to both diagnose tumorigenic cells in either biopsies or other patient samples, and to selectively target tumor-initiating cells for destruction as a therapeutic strategy.

### BRIEF DESCRIPTION OF THE DRAWINGS

FIGS. 1A-B are Venn diagrams showing significantly higher expression of CD24 in all four primary brain tumor types relative to normal glial progenitors. Selective tumor progenitor expressed genes were identified by comparing the profile of A2B5-isolated glioma tumor progenitors to native normal A2B5-isolated glial progenitors from adult human brain. Gliomas were subdivided into four categories, oligodendroglioma (OLG), oligoastrocytoma (OA), astrocytoma (A), and glioblastoma multiforme (GBM). Using a linear-modeling approach, normalized glioma progenitor profiles were directly compared with normal progenitors (A2B5norm). The Venn diagrams illustrate the number of transcripts (Affymetrix probe sets) significantly regulated in each comparison (>3 fold change, t-test 5% false discovery rate). The overlap in regulated transcripts in tumor progenitor cells of oligodendroglioma, oligoastrocytoma, and astrocytoma is shown in FIG. 1A, and the overlap in regulated transcripts of tumor progenitor cells of oligoastrocytoma, astrocytoma, and glioblastoma multiforme is shown in FIG. 1B. CD24 mRNA was among a restricted set of transcripts whose expression was selective to tumor progenitors isolated from every glioma type.

FIG. 2 is a gene expression heatmap of CD24 mRNA expression by microarray analysis in a panel of A2B5-sorted cells isolated from 14 gliomas compared to unsorted (uns), A2B5-selected cells from adult human white matter (WM) or cortex (CTX), and CD11b microglial cells. A=astrocytoma, OLG=Oligodendroglioma, OA=Oligo-astrocytoma, GBM=Glioblastoma, CTX=Cortex, WM=White matter, uns=Unsorted

FIGS. 3A-O are a panel of photographs showing fluorescent immunohistochemical detection of CD24 expression in fetal (FIGS. 3A-I) and adult (FIGS. 3J-O) human brain sections. In the fetal human brain, CD24 is diffusely and intensively expressed from the cortical plate (CP) (FIGS. 3A-C), the intermediate zone (FIGS. 3D-F), and to the sub-ventricular zone (SVZ) (FIGS. 3G-I). A lower degree of immunoreactivity is observed in the ventricular zone (FIGS. 3G-I). Tissue sections were counter stained with DAPI to show individual cell nuclei. VZ=ventricular zone; SVZ=sub-ventricular zone; IZ=intermediate zone; CP=cortical plate. In the adult brain, CD24 expression appears to be restricted to the ependymal layer (EL) with a diffuse pattern observed in the sub-ependymal layer (SEL) (FIGS. 3J-L). FIGS. 3M-N show minimal background control (Ct) staining (omission of primary antibody). FIGS. 3A, D, G, and J show an overlay of CD24 expression and cell nuclear staining (DAPI); FIGS. 3B, E, H, and K show CD24 expression alone; and FIGS. 3C, F, I and L show DAPI labeling alone. Scale bar, 100 μm.

FIGS. 4A-H are a panel of photographs showing fluorescent immunohistochemical detection and co-localization of CD24 and Sox2 expression (FIGS. 4E-H), and CD24 and Musashi-1 (Msi-1) expression (FIGS. 4A-D) in the ventricular zone (VZ) and sub-ventricular zone (SVZ) of fetal human brain. DAPI staining shows individual cell nuclei. Scale bar, 100 μm.

FIGS. 5A-D show fluorescent immunohistochemical detection of CD24 expression in various glioma samples including glioblastoma (GBM) (FIG. 5A), astrocytoma (A) (FIG. 5B), oligodendroglioma (O) (FIG. 5C), and oligoastrocytoma (OA) (FIG. 5D). Scale bar, 100 μm.

FIGS. 6A-F are a panel of fluorescent immunohistochemical photographs illustrating the strong association between CD24 expression and the expression of the stem/progenitor cell marker SOX2 and the oligodendrocytic marker OLIG2 in oligodendroglioma (FIGS. 6A-13), astrocytoma (FIGS. 6C-D), and glioblastoma (FIGS. 6E-F). Scale bar, 100 μm.

FIGS. 7A-L are fluorescent immunohistoehemical photographs showing CD24 expression in association with oligodendrocytic progenitor marker PDGFRa (FIGS. 7A-C), neuronal marker Tuj1 (FIGS. 7D-F), the astrocytic marker GFAP (FIGS. 7G-I) and the stem cell marker Nestin (FIGS. 7J-L) in an oligo-astrocytoma. Scale bar, 100 μm.

FIG. 8 is a bar graph showing the level of CD24 expression in glioblastoma (U87, U373, U251, SID 227) and gliosarcoma (SID 238) cell lines detected by flow cytometry. SF=Serum-free media, FBS=culture media supplemented with fetal-bovine-serum.

### DETAILED DESCRIPTION OF THE INVENTION

A first aspect of the invention relates to a method of treating a primary brain tumor in a subject. This method involves administering to the subject a CD24-specific targeting component under conditions effective to treat the primary brain tumor in the subject. In accordance with this aspect of the invention, a subject having one or more primary brain tumors is selected and the CD24-specific targeting component is administered to the selected subject.

The present invention further relates to a method of preventing the migratory spread of a primary brain tumor in a subject. This method involves administering to the subject a CD24-specific targeting component under conditions effective to prevent the migratory spread of a primary brain tumor within the subject. In accordance with this aspect of the invention, a subject at risk for the migratory spread of one or more primary brain tumors is selected and the CD24-specific targeting component is administered to the selected subject.

The methods of the present invention can be used to treat a primary brain tumor or prevent the migratory spread of a brain tumor in any subject, preferably an animal subject, in need of such treatment. In a preferred embodiment, the subject is a mammalian subject. Exemplary mammalian subjects include, without limitation, humans, non-human primates, dogs, cats, rodents (e.g., mouse, rat, guinea pig), horses, cattle and cows, sheep, and pigs.

As used herein, a primary brain tumor is any tumor that originates in the brain. Primary brain tumors are generally classified and named according to the normal brain cells from which they originate (e.g. astrocytes, oligodendrocytes, neural cells, etc.) or the location in the brain from which they originate (e.g. menengies, cerebrum, brain stem, etc). Primary brain tumors suitable for treatment in accordance with the method of the present invention include, but are not limited to, gliomas, mixed oligo-astrocytomas, glioblastomas, anaplastic astrocytomas, oligodendrogliomas, neurocytomas, primitive neuroectodermal tumors, dysplastic neuroepithelial tumors, and gangliogliomas.

CD24 is a 35-45 kDa GPI-linked membrane sialoglycoprotein that serves as a ligand for p-selectin. The studies described herein have identified overexpression of CD24 on tumor progenitor cells relative to parental tumor cells in various primary brain tumors, including glioblastoma, anaplatic astrocytoma, oligodendroglioma, and neurocytoma. Accordingly, CD24 is a suitable target for primary brain tumor pharmacotherapy, both as a means of directly inhibiting tumor growth and migration as well as a means for targeting oncolytic therapeutics to the tumor site.

The CD24-specific-targeting component of the present invention is meant to encompass any CD24 binding molecule. In a preferred embodiment, the CD24-specific targeting component is also a CD24 antagonist. A CD24 antagonist includes any molecule that partially or fully blocks, inhibits, or neutralizes the biological activity of CD24. Suitable CD24-specific targeting components useful for carrying out the methods of the present invention include, but are not limited to, nucleic acid molecules, proteins or polypeptides, and small molecules.

Exemplary nucleic acid CD24-specific targeting components include antisense RNAs or RNAi, such as short interfering RNAs (siRNA), short hairpin RNAs (shRNA), and microRNAs.

The use of antisense methods to inhibit the in vivo translation of genes and subsequent protein expression is well known in the art (e.g., U.S. Pat. No. 7,425,544 to Dobie et al.; U.S. Pat. No. 7,307,069 to Karras et al.; U.S. Pat. No. 7,288,530 to Bennett et al.; U.S. Pat. No. 7,1791796 to Cowsert et al., which are hereby incorporated by reference in their entirety). Antisense nucleic acids are nucleic acid molecules (e.g., molecules containing DNA nucleotides, RNA nucleotides, or modifications (e.g., modification that increase the stability of the molecule, such as 2′-O-alkyl (e.g., methyl) substituted nucleotides) or combinations thereof) that are complementary to, or that hybridize to, at least a portion of a specific nucleic acid molecule, such as an mRNA molecule (See e.g., Weintraub, H. M., “Antisense DNA and RNA,” Scientific Am. 262:40-46 (1990), which is hereby incorporated by reference in its entirety). The antisense nucleic acids hybridize to corresponding nucleic acids, such as mRNAs, to form a double-stranded molecule, which interferes with translation of the mRNA, as the cell will not translate a double-stranded mRNA. Antisense nucleic acids used in the methods of the present invention are typically at least 10-12 nucleotides in length, for example, at least 15, 20, 25, 50, 75, or 100 nucleotides in length. The antisense nucleic acid can also be as long as the target nucleic acid with which it is intended to form an inhibitory duplex. Antisense nucleic acids can be introduced into cells as antisense oligonucleotides, or can be produced in a cell in which a nucleic acid encoding the antisense nucleic acid has been introduced, for example, using gene therapy methods.

siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of the CD24 mRNA sequence. siRNA molecules are typically designed to target a region of the mRNA target approximately 50-100 nucleotides downstream from the start codon. Upon introduction into a cell, the siRNA complex triggers the endogenous RNA interference (RNAi) pathway, resulting in the cleavage and degradation of the target mRNA molecule. siRNA molecules that effectively interfere with CD24 expression have been developed (e.g., Santa Cruz Biotechnology, Inc.) and are suitable for use in the present invention. In addition, various improvements of siRNA compositions, such as the incorporation of modified nucleosides or motifs into one or both strands of the siRNA molecule to enhance stability, specificity, and efficacy, have been described and are suitable for use in accordance with this aspect of the invention (See e.g., WO 2004/015107 to Giese et al.; WO 2003/070918 to McSwiggen et al.; WO 1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety).

Short or small hairpin RNA molecules are similar to siRNA molecules in function, but comprise longer RNA sequences that make a tight hairpin turn. shRNA is cleaved by cellular machinery into siRNA and silence gene expression via the cellular RNA interference pathway.

Nucleic acid aptamers that specifically bind to CD24 are also useful in the methods of the present invention. Nucleic acid aptamers are single-stranded, partially single-stranded, partially double-stranded, or double-stranded nucleotide sequences, advantageously a replicatable nucleotide sequence, capable of specifically recognizing a selected non-oligonucleotide molecule or group of molecules by a mechanism other than Watson-Crick base pairing or triplex formation. Aptamers include, without limitation, defined sequence segments and sequences comprising nucleotides, ribonucleotides, deoxyribonucleotides, nucleotide analogs, modified nucleotides, and nucleotides comprising backbone modifications, branchpoints, and non-nucleotide residues, groups, or bridges. Nucleic acid aptamers include partially and fully single-stranded and double-stranded nucleotide molecules and sequences; synthetic RNA, DNA, and chimeric nucleotides; hybrids; duplexes; heteroduplexes; and any ribonucleotide, deoxyribonucleotide, or chimeric counterpart thereof and/or corresponding complementary sequence, promoter, or primer-annealing sequence needed to amplify, transcribe, or replicate all or part of the aptamer molecule or sequence.

As described supra, the CD24-specific targeting component can also be a protein or polypeptide CD24-binding molecule. Exemplary protein or polypeptide CD24 binding molecules preferably have little or no binding affinity for non-CD24 proteins.

In accordance with this aspect of the invention, the CD24-specific targeting component may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. CD24 binding molecules may have both a heavy and a light chain. Preferred CD24-specific targeting components of the present invention include, antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, so long as they exhibit the desired activity, e.g., binding to CD24.

CD24 antibodies suitable for use in the methods of the present invention are known in the art (See e.g. Sagiv et al., “Targeting CD24 for Treatment of Colorectal and Pancreatic Cancer by Monoclonal Antibodies or Interfering RNA,” Cancer Research 68:2803-2812 (2008); Benkerrou et al., “Anti-B-Cell Monoclonal Antibody Treatment of Severe Posttransplant B-Lymphorproliferative Disorder: Prognostic Factors and Long-Term Outcome,” Blood 92(9):3137-47 (1998); Fischer et al., “Anti-B-Cell Monoclonal Antibodies in the Treatment of Severe B-Cell Lymphoproliferative Syndrome Following Bone Marrow and Organ Transplantation,” N Engl J Med 324(21):1452-6 (1991), which are hereby incorporated by reference in their entirety). Alternatively, anti-CD24 antibodies can be produced using any of the commonly utilized methods for generating antibodies known to those in the art.

Procedures for raising polyclonal antibodies are well known in the art Typically, such antibodies are raised by immunizing an animal (e.g. a rabbit, rat, mouse, donkey, etc) by multiple subcutaneous or intraperitoneal injections of the relevant antigen (a purified CD24 peptide fragment, full-length recombinant CD24 protein, fusion protein, etc) optionally conjugated to keyhole limpet hemocyanin (KLH), serum albumin, other immunogenic carrier, diluted in sterile saline and combined with an adjuvant (e.g. Complete or Incomplete Freund's Adjuvant) to form a stable emulsion. The polyclonal antibody is then recovered from blood or ascites of the immunized. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc. Polyclonal antiserum can also be rendered monospecific using standard procedures (See e.g. Agaton et al., “Selective Enrichment of Monospecific Polyclonal Antibodies for Antibody-Based Proteomics Efforts,” J Chromatography A 1043(1):33-40 (2004), which is hereby incorporated by reference in its entirety).

Monoclonal antibodies can be prepared using hybridoma methods, such as those described by Kohler and Milstein, “Continuous Cultures of Fused Cells Secreting Antibody of Predefined Specificity,” Nature 256:495-7 (1975), which is hereby incorporated by reference in its entirety. Using the hybridoma method, a mouse, hamster, or other appropriate host animal, is immunized to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against CD24, as determined by immunoprecipitation, immunoblotting, or by an in vitro binding assay such as radioimmunoassay (RIA) or enzyme-linked immunosorbent assay (ELISA) can then be propagated either in vitro culture using standard methods (James Goding, MONOCLONAL ANTIBODIES: PRINCIPLES AND PRACTICE (1986) which is hereby incorporated by reference in its entirety) or in vivo as ascites tumors in an animal. The monoclonal antibodies can then be purified from the culture medium or ascites fluid as described for polyclonal antibodies above.

Alternatively, monoclonal antibodies can be made using recombinant DNA methods as described in U.S. Pat. No. 4,816,567 to Cabilly et al, which is hereby incorporated by reference in its entirety. The polynucleotides encoding a monoclonal antibody are isolated, such as from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, and monoclonal antibodies are generated by the host cells. Recombinant monoclonal antibodies or fragments thereof of the desired species can also be isolated from phage display libraries as described (McCafferty et al., “Phage Antibodies: Filamentous Phage Displaying Antibody Variable Domains,” Nature 348:552-554 (1990); Clackson et al., “Making Antibody Fragments using Phage Display Libraries,” Nature 352:624-628 (1991); and Marks et al., “By-Passing Immunization. Human Antibodies from V-Gene Libraries Displayed on Phage,” J. Mol. Biol. 222:581-597 (1991), which are hereby incorporated by reference in their entirety).

The polynucleotide(s) encoding a monoclonal antibody can further be modified in a number of different ways using recombinant DNA technology to generate alternative antibodies. In one embodiment, the constant domains of the light and heavy chains of, for example, a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

In some embodiments of the present invention, the monoclonal antibody against CD24 is a humanized antibody. Humanized antibodies are antibodies that contain minimal sequences from non-human (e.g. murine) antibodies within the variable regions. Such antibodies are used therapeutically to reduce antigenicity and human anti-mouse antibody responses when administered to a human subject. In practice, humanized antibodies are typically human antibodies with minimum to no non-human sequences. A human antibody is an antibody produced by a human or an antibody having an amino acid sequence corresponding to an antibody produced by a human.

Humanized antibodies can be produced using various techniques known in the art. An antibody can be humanized by substituting the complementarity determining region (CDR) of a human antibody with that of a non-human antibody (e.g. mouse, rat, rabbit, hamster, etc.) having the desired specificity, affinity, and capability (Jones et al., “Replacing the Complementarity-Determining Regions in a Human Antibody With Those From a Mouse,” Nature 321:522-525 (1986); Riechmann et al., “Reshaping Human Antibodies for Therapy,” Nature 332:323-327 (1988); Verhoeyen et al., “Reshaping Human Antibodies: Grafting an Antilysozyme Activity,” Science 239:1534-1536 (1988), which are hereby incorporated by reference in their entirety). The humanized antibody can be further modified by the substitution of additional residues either in the Fv framework region and/or within the replaced non-human residues to refine and optimize antibody specificity, affinity, and/or capability.

Human antibodies can be directly prepared using various techniques known in the art. Immortalized human B lymphocytes immunized in vitro or isolated from an immunized individual that produce an antibody directed against a target antigen can be generated (See e.g. Reisfeld et al., Monoclonal Antibodies and Cancer Therapy 77 (Alan R. Liss 1985) and U.S. Pat. No. 5,750,373 to Garrard, which are hereby incorporated by reference in their entirety). Also, the human antibody can be selected from a phage library, where that phage library expresses human antibodies (Vaughan et al., “Human Antibodies with Sub-Nanomolar Affinities Isolated from a Large Non-immunized Phage Display Library,” Nature Biotechnology, 14:309-314 (1996); Sheets et al., “Efficient Construction of a Large Nonimmune Phage Antibody Library: The Production of High-Affinity Human Single-Chain Antibodies to Protein Antigens,” Proc Nat'l Acad Sci USA 95:6157-6162 (1998); Hoogenboom et al., “By-passing Immunisation. Human Antibodies From Synthetic Repertoires of Germline VH Gene Segments Rearranged In Vitro,” J Mol. Biol, 227:381-8 (1992); Marks et al., “By-passing Immunization. Human Antibodies from V-gene Libraries Displayed on Phage,” J. Mol. Biol, 222:581-97 (1991), which are hereby incorporated by reference in their entirety). Humanized antibodies can also be made in transgenic mice containing human immunoglobulin loci that are capable upon immunization of producing the full repertoire of human antibodies in the absence of endogenous immunoglobulin production. This approach is described in U.S. Pat. No. 5,545,807 to Surani et al.; U.S. Pat. No. 5,545,806 to Lonberg et al.; U.S. Pat. No. 5,569,825 to Lonberg et al.; U.S. Pat. No. 5,625,126 to Lonberg et al.; U.S. Pat. No. 5,633,425 to Lonberg et al.; and U.S. Pat. No. 5,661,016 to Lonberg et al., which are hereby incorporated by reference in their entirety

Bispecific antibodies that specifically recognize CD24 are also suitable for use in the methods of the present invention. Bispecific antibodies are antibodies that are capable of specifically recognizing and binding at least two different epitopes. Bispecific antibodies can be intact antibodies or antibody fragments. Techniques for making bispecific antibodies are common in the art (Brennan et al., “Preparation of Bispecific Antibodies by Chemical Recombination of Monoclonal Immunoglobulin G1 Fragments,” Science 229:81-3 (1985); Suresh et al, “Bispecific Monoclonal Antibodies From Hybrid Hybridomas,” Methods in Enzymol. 121:210-28 (1986); Traunecker et al., “Bispecific Single Chain Molecules (Janusins) Target Cytotoxic Lymphocytes on HIV Infected Cells,” EMBO J. 10:3655-3659 (1991); Shalaby et al., “Development of Humanized Bispecific Antibodies Reactive with Cytotoxic Lymphocytes and Tumor Cells Overexpressing the HER2 Protooncogene,” J. Exp. Med. 175:217-225 (1992); Kostelny et al, “Formation of a Bispecific Antibody by the Use of Leucine Zippers,” J. Immunol. 148: 1547-1553 (1992); Gruber et al., “Efficient Tumor Cell Lysis Mediated by a Bispecific Single Chain Antibody Expressed in Escherichia coli,” J. Immunol. 152:5368-74 (1994); and U.S. Pat. No. 5,731,168 to Carter et al., which are hereby incorporated by reference in their entirety).

In certain embodiments of the present invention, it may be desirable to use an antibody fragment, rather than an intact antibody, to increase tumor penetration. Various techniques are known for the production of antibody fragments. Traditionally, these fragments are derived via proteolytic digestion of intact antibodies (e.g. Morimoto et al., “Single-step Purification of F(ab′)2 Fragments of Mouse Monoclonal Antibodies (immunoglobulins G1) by Hydrophobic Interaction High Performance Liquid Chromatography Using TSKgel Phenyl-5PW,” Journal of Biochemical and Biophysical Methods 24:107-117 (1992) and Brennan et al., “Preparation of Bispecific Antibodies by Chemical Recombination of Monoclonal Immunoglobulin G1 Fragments,” Science 229:81-3 (1985), which are hereby incorporated by reference in their entirety). However, these fragments are now typically produced directly by recombinant host cells as described above. Thus Fab, Fv, and scFv antibody fragments can all be expressed in and secreted from E. coli or other host cells, thus allowing the production of large amounts of these fragments. Alternatively, such antibody fragments can be isolated from the antibody phage libraries discussed above. The antibody fragment can also be linear antibodies as described in U.S. Pat. No. 5,641,870 to Rinderknecht et al., which is hereby incorporated by reference, and can be monospecific or bispecific. Other techniques for the production of antibody fragments will be apparent to the skilled practitioner.

It may further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

The present invention further encompasses variants and equivalents which are substantially homologous to the chimeric, humanized and human antibodies, or antibody fragments thereof. These can contain, for example, conservative substitution mutations, i.e. the substitution of one or more amino acids by similar amino acids, which maintain or improve the binding activity of the antibody or antibody fragment.

In addition to antibodies and antibody fragments, other suitable CD24-specific targeting components of the present invention include antibody mimics and CD24 binding oligopeptides.

A number of antibody mimics are known in the art including, without limitation, those known as monobodies, which are derived from the tenth human fibronectin type III domain (¹⁰Fn3) (Koide et al., “The Fibronectin Type III Domain as a Scaffold for Novel Binding Proteins,” J. Mol. Biol. 284:1141-1151 (1998); Koide et al., “Probing Protein Conformational Changes in Living Cells by Using Designer Binding Proteins: Application to the Estrogen Receptor,” Proc. Nat'l Acad. Sci. USA 99:1253-1258 (2002), which are hereby incorporated by reference in their entirety); and those known as affibodies, which are derived from the stable α-helical bacterial receptor domain Z of staphylococcal protein A (Nord et al., “Binding Proteins Selected from Combinatorial Libraries of an a-Helical Bacterial Receptor Domain,” Nat. Biotechnol. 15(8):772-777 (1997), which is hereby incorporated by reference in its entirety). Variations in these antibody mimics can be created by substituting one or more domains of these polypeptides with a CD24 specific domain and then screening the modified monobodies or affibodies for specificity for binding to CD24.

A CD24-binding oligopeptide is an oligopeptide that binds, preferably specifically to the CD24 protein. Such oligopeptides may be chemically synthesized using known oligopeptide synthesis methodology or may be prepared and purified using recombinant technology. Such oligopeptides are usually at least about 5 amino acids in length, but can be anywhere from 5 to 100 amino acids in length. Such oligopeptides may be identified without undue experimentation using well known techniques. Techniques for screening oligopeptide libraries for oligopeptides that are capable of specifically binding to a polypeptide target, in this case CD24, are well known in the art (see e.g. U.S. Pat. No. 5,556,762 to Pinilla et al.; U.S. Pat. No. 5,750,373 to Garrard et al.; U.S. Pat. No. 4,708,871 to Geysen; U.S. Pat. No. 4,833,092 to Geysen; U.S. Pat. No. 5,223,409 to Ladner et al.; U.S. Pat. No. 5,403,484 to Ladner et al.; U.S. Pat. No. 5,571,689 to Heuckeroth et al.; U.S. Pat. No. 5,663,143 to Ley et al.; and PCT Publication Nos. WO84/03506 to Geysen and WO84/03564 Geysen, which are hereby incorporated by reference in their entirety).

In one embodiment of the present invention the CD24-specific targeting component is linked or conjugated to a cancer therapeutic agent to facilitate direct delivery of the therapeutic agent to the primary brain tumor. In a preferred embodiment, the CD24-specific targeting component is an antibody that is linked or conjugated to a cancer therapeutic. Methods of making such conjugates, in particular antibody-drug conjugates, are known in the art and are described in WO2005/077090 to Duffy et al., WO2005/082023 to Feng, WO2005/084390 to Alley et al., WO2006/065533 to McDonagh et al., WO2007/103288 to McDonagh et al., WO2007/011968 to Jeffery and WO2008/070593 to McDonagh et al., which are all hereby incorporated by reference in their entirety. Cancer therapeutics that can be linked to the CD24-specific targeting component include, but are not limited to, chemotherapeutic agents, radioisotopes, or immunotherapeutic agents.

In accordance with this aspect of the invention, exemplary chemotherapeutic agents include the toxins, diphtheria, ricin, and cholera toxin. Other chemotherapeutic agents that can be linked to the CD24-specific targeting component of the present invention include alkylating agents (e.g. cisplatin, carboplatin, oxaloplatin, mechlorethamine, cyclophosphamide, chorambucil, nitrosureas); anti-metabolites (e.g. methotrexate, pemetrexed, 6-mercaptopurine, dacarbazine, fludarabine, 5-fluorouracil, arabinosycytosine, capecitabine, gemcitabine, decitabine); plant alkaloids and terpenoids including vinca alkaloids (e.g. vincristine, vinblastine, vinorelbine), podophyllotoxin (e.g. etoposide, teniposide), taxanes (e.g. paclitaxel, docetaxel); topoisomerase inhibitors (e.g. notecan, topotecan, amasacrine, etoposide phosphate); antitumor antibiotics (dactinomycin, doxorubicin, epirubicin, and bleomycin); ribonucleotides reductase inhibitors; antimicrotubules agents; and retinoids.

In an alternative embodiment of the present invention, the CD24-specific targeting component is linked to a radioisotope. Exemplary radiotherapeutic agents include any nuclide emitting radioactive ray usable for the cancer treatment, including x-ray, gamma-ray, electron beam, photon, alpha-particle and neutron, etc. The above nuclide is exemplified by ¹³¹I, ⁶⁰Co, ⁵⁷Co, ¹⁹²Ir, ¹⁶⁶Ho, ³²P, ⁴⁸V, ¹⁹⁸Au, ^{99m}Tc, ¹²⁵I, ¹⁶⁵Dy, ¹⁸⁸Re, ¹⁶⁹Er, ¹⁵³Sm, ⁹⁰Y, ¹⁰⁹Pd, and ⁸⁹Sr. Such radioactive rays are preferably in complex with a carrier such as chitosan to prevent leakage from the tumor lesion to the surrounding tissue.

In another embodiment, the CD24-specific targeting component of the present invention is linked to an immunotherapeutic agent. The immunotherapeutic agent can be a cytokine. The cytokine is exemplified by interleukin-1 (IL-I), IL-2, IL-4, IL-5, IL-β, IL-7, IL-10, IL-12, IL-I5, IL-18, CSF-GM, CSF-G, IFN-γ, IFN-α, TNF, TGF-β, FLT-3 ligand and CD40 ligand, but not always limited thereto.

In another embodiment of the present invention, the CD24-specific targeting component can be linked or conjugated to a delivery vehicle containing a cancer therapeutic. Suitable delivery vehicles include liposomes (Hughes et al., “Monoclonal Antibody Targeting of Liposomes to Mouse Lung In Vivo,” Cancer Res 49(22):6214-20 (1989), which is hereby incorporated by reference in its entirety), nanoparticles (Farokhzad et al., “Targeted Nanoparticle-Aptamer Bioconjugates for Cancer Chemotherapy In Viva,” Proc Nat'l Acad Sci USA 103(16):6315-20(2006), which is hereby incorporated by reference in its entirety), biodegradable microspheres, microparticles, and collagen minipellets. The delivery vehicle can contain any of the chemotherapeutic, radiotherapeutic, or immunotherapeutic agents described supra.

In one embodiment, the CD24-specific targeting component is conjugated to a liposome delivery vehicle (Sofou & Sgouros, “Antibody-Targeted Liposomes in Cancer Therapy and Imaging,” Exp Opin Drug Deliv 5(2):189-204 (2008), which is hereby incorporated by reference in its entirety). Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug (cancer therapeutic) at the primary brain tumor site. This can be accomplished, for example, in a passive manner where the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Wang & Huang, “pH-Sensitive Immunoliposomes Mediate Target-cell-specific Delivery and Controlled Expression of a Foreign Gene in Mouse,” Proc. Nat'l Acad. Sci. USA 84:7851-5 (1987), which is hereby incorporated by reference in its entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane, which enzyme slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

Different types of liposomes can be prepared according to Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; and U.S. Pat. No. 5,059,421 to Loughrey et al., which are hereby incorporated by reference in their entirety.

These liposomes can be produced such that they contain, in addition to the therapeutic agents of the present invention, other therapeutic agents, such as immunotherapeutic cytokines, which would then be released at the target site (e.g., Wolff et al., “The Use of Monoclonal Anti-Thy1 IgG1 for the Targeting of Liposomes to AKR-A Cells in Vitro and in Vivo,” Biochim. Biophys. Acta 802:259-73 (1984), which is hereby incorporated by reference in its entirety).

In accordance with the methods of the present invention, administering to a subject having a primary brain tumor the CD24-specific targeting component alone, linked to a cancer therapeutic agent, or conjugated to a delivery vehicle containing a cancer therapeutic, causes a reduction in the number of tumor cells, a reduction in tumor growth, and a reduction in tumor size or bulk. In one embodiment, administration of the CD24-specific targeting component diminishes tumor invasion and migration (i.e. tumor metastasis) thereby delaying or inhibiting tumor progression. Administration of the CD24-specific targeting component alone or linked to a cancer therapeutic agent alleviates one or more of the symptoms associated with the specific primary brain tumor and reduces or prevents morbidity and mortality of the subject having the primary brain tumor. Preferred methods and mode of administration are described infra.

Another aspect of the present invention relates to pharmaceutical compositions comprising the CD24-specific targeting component alone, the CD-specific targeting component conjugated to a cancer therapeutic agent, and/or the CD24-specific targeting component linked to a delivery vehicle, which are suitable for treating a primary brain tumor or preventing its migratory spread. Therapeutic formulations of the CD24-specific targeting component (e.g. CD24 antibodies or antibody fragments, binding oligopeptides, CD24 RNAi or antisense molecules, and CD24 binding small molecules) are prepared for storage by mixing the antibody, oligopeptide, nucleic acid or small molecule having the desired degree of purity with optional pharmaceutically acceptable carriers, excipients or stabilizers (REMINGTON'S PHARMACEUTICAL SCIENCES (A. Osol ed. 1980), which is hereby incorporated by reference in its entirety), in the form of lyophilized formulations or aqueous solutions. Acceptable carriers, excipients, or stabilizers are nontoxic to recipients at the dosages and concentrations employed, and include buffers such as acetate, Tris-phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG).

The formulations herein may also contain more than one active compound as necessary for the particular indication being treated, preferably those with complementary activities that do not adversely affect each other. For example, in addition to a CD24 binding oligopeptide, CD24 RNAi, or CD24 binding small molecule, it may be desirable to include in the one formulation, an additional RNAi, e.g., a second CD24 RNAi which binds a different area on the CD24 nucleic acid, or to some other target such as a growth factor that affects the growth of the particular brain tumor. Alternatively, or additionally, the composition may further comprise a chemotherapeutic agent, cytotoxic agent, cytokine, growth inhibitory agent, anti-hormonal agent, and/or cardioprotectant. Such molecules are suitably present in combination in amounts that are effective for the purpose intended.

The active therapeutic ingredients of the pharmaceutical compositions (i.e. CD24-specific targeting component alone or linked to a cancer therapeutic agent) can be entrapped in microcapsules prepared using coacervation techniques or by interfacial polymerization, e.g., hydroxymethylcellulose or gelatin-microcapsules and poly-(methylmethacylate) microcapsules, respectively, in colloidal drug delivery systems (e.g., liposomes, albumin microspheres, microemulsions, nano-particles and nanocapsules) or in macroemulsions. Such techniques are disclosed in REMINGTON'S PHARMACEUTICAL SCIENCES (A. Osol ed. 1980), which is hereby incorporated by reference in its entirety. As described supra, the CD24-specific targeting component can also be conjugated to the microcapsule delivery vehicle to target the delivery of the therapeutic agent to the site of the tumor. Sustained-release preparations may be prepared. Suitable examples of sustained-release preparations include semi-permeable matrices of solid hydrophobic polymers containing the antibody or polypeptide, which matrices are in the form of shaped articles, e.g., films or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid.

The formulations to be used for in viva administration must be sterile. This is readily accomplished by filtration through sterile filtration membranes.

The therapeutically effective compositions containing the CD24-specific targeting component of the present invention are administered to a subject, in accordance with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Systemic, intravascular, intraventricular, and intraparenchymal administration of the CD24-specific targeting component to the brain, brainstem, or spinal cord is preferred. In an alternative embodiment, compositions comprising the CD24-specific targeting component are administered using convection enhanced delivery (CED). CED is a method which involves direct perfusion of the central nervous system interstitial spaces using convective force that is achieved by a slight hydrostatic pressure generated by a syringe pump. CED allows for safe, targeted, homogenous delivery of therapeutic agents, including therapeutic agents encapsulated in liposomes or other delivery vehicles, into small and large tissue volumes without interference from the blood brain barrier. CED methodology is well known in the art (See e.g. Saito et al., “Distribution of Liposomes into Brain and Rat Brain Tumor Models by Convection-Enhanced Delivery Monitored with magnetic Resonance Imaging,” Cancer Research 64:2572-2579 (2004); Vogelbaum M., “Convention Enhanced Delivery for the Treatment of Malignant Gliomas: Symposium Review,” J Neuro-Oncology 73(1):57-69 (2005); Perlstein et al., “Convection-Enhanced Delivery of Maghemite Nanoparticles: Increased Efficacy and MRI Monitoring,” Neuro-Oncol 10(20):153-161 (2008); Krauze et al., “Convection-Enhanced Delivery of Nanoliposomal CPT-11 (Irinotecan) and PEGylated Liposomal Doxorubicin (Doxil) in Rodent Intracranial Brain Tumor Xenografts,” Neuro-Oncol 9(4):393-403 (2007), which are all hereby incorporated by reference in their entirety).

Other therapeutic regimens may be combined with the administration of the CD24-specific targeting component. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

It may also be desirable to combine administration of the CD24 targeting component, with administration of an antibody directed against another tumor antigen associated with the primary brain tumor.

In another embodiment, the therapeutic treatment methods of the present invention involve the combined administration of one or more CD24-specific targeting components, each component conjugated to a distinct chemotherapeutic agent, radiotherapeutic agent, or immunotherapeutic agent, resulting in the administration of a cocktail of chemotherapeutic, radiotherapeutic, and/or immunotherapeutic agents. In another embodiment, the CD24-specific targeting component alone or conjugated to the cancer therapeutic can be administered with one or more additional chemotherapeutic agents. Preparation and dosing schedules for such chemotherapeutic agents may be used according to manufacturers' instructions or as determined empirically by the skilled practitioner. Preparation and dosing schedules for such chemotherapy are also described in CHEMOTHERAPY SERVICE (M. C. Perry ed., 1992), which is hereby incorporated by reference in its entirety.

Sometimes, it may be beneficial to also co-administer a cardioprotectant (to prevent or reduce myocardial dysfunction associated with the therapy) or one or more cytokines to the patient. In addition to the above therapeutic regimes, the patient may be subjected to surgical removal of cancer cells and/or radiation therapy, before, simultaneously with, or post CD24-specific targeting component therapy. Suitable dosages for any of the above co-administered agents are those presently used and may be lowered due to the combined action (synergy) of the agent and CD24-specific targeting component.

For the treatment of a primary brain tumor, the dosage and mode of administration will be chosen by the physician according to known criteria. A therapeutically effective dose of the CD24-specific targeting component alone or linked to a cancer therapeutic agent is the amount effective for reducing tumor cells, reducing tumor size, reducing tumor cell migration and invasion, or reducing tumor growth. The dosage should not cause adverse side effects, such as unwanted cross-reactions, anaphylactic reactions, and the like. Generally, the dosage will vary with the age, condition, sex and route of administration, or whether other drugs are included in the regimen, and can be determined by one of skill in the art. The appropriate dosage of the CD24-specific targeting component will also depend on the type of primary brain tumor to be treated and the severity and course of the disease. The CD24-specific targeting component is suitably administered to the patient at one time or over a series of treatments. Preferably, the CD24-specific targeting component is administered by intravenous infusion or by subcutaneous injections. Depending on the type and severity of the tumor, about 1 μg/kg to about 50 mg/kg body weight (e.g., about 0.1-15 mg/kg/dose) of a CD24 antibody can be an initial candidate dosage for administration to the subject, whether, for example, by one or more separate administrations, or by continuous infusion. A dosing regimen can comprise administering an initial loading dose of about 4 mg/kg, followed by a weekly maintenance dose of about 2 mg/kg of the anti-CD24 antibody. However, other dosage regimens may be useful. A typical daily dosage might range from about 1 μg/kg to 100 mg/kg or more, depending on the factors mentioned above. Further guidance in selecting appropriate doses for antibodies is found in the HANDBOOK OF MONOCLONAL ANTIBODIES 303-357 (Ferrone et al. eds., 1985) and Smith et al., ANTIBODIES IN HUMAN DIAGNOSIS AND THERAPY 365-389 (Haber et al. eds., 1977), which are hereby incorporated by reference in their entirety.

For repeated administrations over several days or longer, depending on the condition, the treatment is sustained until a desired suppression of tumor growth occurs. The progress of this therapy can be readily monitored by conventional methods, assays, and criteria known in the art and described below.

Nucleic acid CD24-specific targeting components of the present invention (i.e. CD24 RNAi, antisense molecules, or apatmers) can be administered to the subject systemically or locally. Delivery of inhibitory RNA is preferably administered alone or as a component of a composition of the present invention. Suitable compositions include the siRNA formulated or complexed with polyethylenimine (e.g., linear or branched PEI) and/or polyethylenimine derivatives, including for example grafted PEIs such as galactose PEI, cholesterol PEI, antibody derivatized PEI, and polyethylene glycol PEI derivatives thereof (see, e.g., Blazek-Welsh & Rhodes, “Maltodextrin-based Proniosomes,” AAPS Pharm. Sci. 3(1):1-11 (2001); Furgeson et al., “Modified Linear Polyethylenimine-cholesterol Conjugates for DNA Complexation,” Bioconjug. Chem. 14:840-7 (2003); Kunath et al., “The Structure of PEG-modified Poly(Ethylene Imines) Influences Biodistribution and Pharmacokinetics of Their Complexes with NF-κB Decoy in Mice,” Pharm. Res. 19:810-7 (2002); Choi et al., “Effect of Poly(Ethylene Glycol) Grafting on Polyethylenimine as a Gene Transfer Vector in Vitro,” Bull. Korean Chem. Soc. 22(1):46-52 (2001); Bettinger et al., “Size Reduction of Galactosylated PEI/DNA Complexes Improves Lectin-mediated Gene Transfer into Hepatocytes,” Bioconjug. Chem. 10:558-61 (1999); Petersen et al., “Polyethylenimine-graft-poly(ethylene glycol) Copolymers: Influence of Copolymer Block Structure on DNA Complexation and Biological Activities as Gene Delivery System,” Bioconjug. Chem. 13:845-54 (2002); Erbacher et al., “Transfection and Physical Properties of Various Saccharide, Poly(Ethylene Glycol), and Antibody-derivatized Polyethylenimines (PEI),” J. Gene Med. 1(3):210-22 (1999); Godbey et al., “Tracking the Intracellular Path of Poly(Ethylenimine)/DNA Complexes for Gene Delivery,” Proc. Nat'l Acad. Sci. USA 96:5177-81 (1999); Godbey et al., “Poly(Ethylenimine) and Its Role in Gene Delivery,” J. Control. Release 60:149-60 (1999); Diebold et al., “Mannose Polyethylenimine Conjugates for Targeted DNA Delivery into Dendritic Cells,” J. Biol. Chem. 274:19087-94 (1999); Thomas & Klibanov, “Enhancing Polyethylenimine's Delivery of Plasmid DNA into Mammalian Cells,” Proc. Nat'l Acad. Sci. USA 99:14640-5 (2002); U.S. Pat. No. 6,586,524 to Sagara, which are hereby incorporated by reference in their entirety).

The CD24-specific targeting nucleic acid molecule can also be present in the form of a bioconjugate, for example a nucleic acid conjugate as described in U.S. Pat. No. 6,528,631 to Cook et al., U.S. Pat. No. 6,335,434 to Guzaev et al., U.S. Pat. No. 6,235,886 to Manoharan & Cook, U.S. Pat. No. 6,153,737 to Manoharan et al., U.S. Pat. No. 5,214,136 to Lin & Matteucci, and U.S. Pat. No. 5,138,045 to Cook & Guinosso, which are hereby incorporated by reference in their entirety.

The CD24-specific targeting nucleic acid molecule, or any composition or bioconjugate containing the same, can be administered via a liposomal delivery mechanism described above.

A third aspect of the present invention relates to a method of diagnosing the existence of a brain tumor in a subject. This method involves providing a patient sample and analyzing the patient sample for the presence of a CD24 antigen or a CD24-encoding nucleic acid molecule, thereby indicating the potential presence of a brain tumor.

Analysis of the patient sample for the presence of the CD24 antigen or a CD24-encoding nucleic acid molecule can be carried out using any molecular probe capable of binding or hybridizing to CD24 RNA, cDNA, or protein to detect and monitor its expression in a sample from the subject. In one embodiment the presence of the CD24 in a patient sample is analyzed using a CD24 nucleic acid probe which binds to CD24 RNA or cDNA in a hybridization reaction. In such an embodiment, it may be desirable to include an amplification reaction prior to, or in conjunction with, the hybridization reaction (e.g. real-time PCR reaction). In a preferred embodiment, the presence of the CD24 antigen in a patient sample is analyzed using an anti-CD24-specific antibody which binds to CD24 in an immunological reaction.

Classical immunohistological methods known to those of skill in the art can be used to analyze the presence of CD24 in a patient sample using a CD24-specific targeting component (e.g. CD24 antibody) (see e.g., Jalkanen et al., “Heparan Sulfate Proteoglycans From Mouse Mammary Epithelial Cells: Localization on the Cell Surface With a Monoclonal Antibody,” J Cell Biol 101:976-84 (1985); Jalkanen et al., “Cell Surface Proteoglycan of Mouse Mammary Epithelial Cells is Shed by Cleavage of its Matrix-Binding Ectodomain from its Membrane-associated Domain,” J Cell Biol 105:3087-96 (1987), which are hereby incorporated by reference in their entirety). Typical immunoassays include, but are not limited to, enzyme linked immunosorbent assay (ELISA), radioimmunoassay (RIA), immunocytochemistry, immunohistochemistry, and western blot. Detection of CD24-specific targeting component binding is facilitated by coupling the targeting component to a detectable substance. Detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the CD24-specific targeting component or indirectly, through an intermediate (such as, for example, a linker known in the art) using techniques known in the art.

Exemplary radioactive labels include ¹²⁵I, ¹³¹I, ¹¹¹In, or ⁹⁹TC. Methods of radiolabeling targeting components, e.g. antibodies or antibody fragments, are known in the art and described in U.S. Pat. No. 5,514,363 to Shochat et al., which is hereby incorporated by reference in its entirety. Radioactivity is detected and quantified using a scintillation counter or autoradiography.

Exemplary fluorescent labels for CD24-specific targeting component detection include chelates (europium chelates), fluorescein and its derivatives, rhodamine and its derivatives, dansyl, Lissamine, phycoerythrin and Texas Red. The fluorescent labels can be conjugated to the CD24-specific targeting component using the techniques disclosed in CURRENT PROTOCOLS IN IMMUNOLOGY (Coligen et al. eds., 1991), which is hereby incorporated by reference in its entirety. Fluorescence can be detected and quantified using a fluorometer.

Suitable enzymatic labels generally catalyze a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Examples of suitable enzymatic labels include luciferases (e.g., firefly luciferase and bacterial luciferase; U.S. Pat. No. 4,737,456 to Weng et al., which is hereby incorporated by reference in its entirety), luciferin, 2,3-dihydrophthalazinediones, malate dehydrogenase, urease, peroxidases (e.g. horseradish peroxidase), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (e.g. uricase and xanthine oxidase), lactoperoxidase, microperoxidase, and the like. Techniques for conjugating enzymes to targeting components, e.g. an antibody, are described in O'Sullivan et al., Methods for the Preparation of Enzyme-Antibody Conjugates for Use in Enzyme Immunoassay, in METHODS IN ENZYMOLOGY 147-66 (Langone et al. eds., 1981), which is hereby incorporated by reference in its entirety.

The label can be indirectly conjugated with the CD24-specific targeting component. For example, the CD24-specific targeting component can be conjugated with biotin and any of the detectable labels mentioned above can be conjugated with avidin, or vice versa. Biotin binds selectively to avidin and thus, the label can be conjugated with the CD24-specific targeting component in this indirect manner. Alternatively, to achieve indirect conjugation of the label with the CD24-specific targeting component, the targeting component is conjugated with a small hapten (e.g., digloxin) and one of the different types of labels mentioned above is conjugated with an anti-hapten antibody (e.g., anti-digloxin antibody). Thus, indirect conjugation of the label with the CD24-specific targeting component can be achieved. In another embodiment, the CD24-specific targeting component is not labeled, and the presence is detected using a labeled antibody which binds to the CD24-specific targeting component.

Another aspect of the present invention relates to a method of diagnosing the existence or monitoring the status of a brain tumor in a subject. This method involves providing a subject and analyzing the subject for the presence or anatomical distribution of a brain tumor based on the presence of a CD24 antigen thereby diagnosing the existence of or monitoring the status of a brain tumor in the subject.

This aspect of the present invention involves administering (for example, parenterally, subcutaneously, or intraperitoneally) a labeled CD24-specific targeting component (e.g. a labeled CD24 antibody or other binding molecule or a CD24 antibody targeted liposome carrying an appropriate label) to the subject, permitting the labeled molecule to preferentially concentrate at sites in the subject where the CD24 antigen is expressed (and for unbound labeled molecule to be cleared to background level), and detecting the amount or level of labeled CD24 specific targeting component bound to the CD24 antigen in the subject after a suitable time interval following the administration. Background level can be determined by various methods including, comparing the amount of labeled molecule detected to a standard value previously determined for a particular system. The size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody or antibody fragment will then preferentially accumulate at the location of cells which contain the CD24 protein. In vivo imaging is described in Burchiel et al., Immunopharmacokinetics of Radiolabeled Antibodies and Their Fragments in TUMOR IMAGING: THE RADIOCHEMICAL DETECTION OF CANCER (Burchiel et al. eds., 1982), which is hereby incorporated by reference in its entirety.

Depending on several variables, including the type of label used and the mode of administration, the time interval following the administration for permitting the labeled molecule to preferentially concentrate at sites in the subject and for unbound labeled molecule to be cleared to background level is 6 to 48 hours, 6 to 24 hours, or 6 to 12 hours. In another embodiment the time interval following administration is 5 to 20 days or 5 to 10 days.

In one embodiment, monitoring the status of the primary brain tumor is carried out by repeating the method for diagnosing the presence of the brain tumor, for example, one month after initial diagnosis, six months after initial diagnosis, one year after initial diagnosis, etc. Monitoring the status of the primary brain tumor is particularly suitable when determining the responsiveness of the tumor to a particular course of therapeutic treatment.

The presence of the labeled molecule can be detected in the patient using methods known in the art for in vivo scanning and imaging of the brain. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography. In one embodiment, the CD24-specific targeting component is labeled with a radioisotope and is detected in the patient using a radiation responsive surgical instrument (U.S. Pat. No. 5,441,050 to Thurston et al., which is hereby incorporated by reference in its entirety). In another embodiment, the CD24-specific targeting component is labeled with a fluorescent compound and is detected in the patient using a fluorescence responsive scanning instrument. In another embodiment, the CD24-specific targeting component is labeled with a positron emitting metal and is detected in the patient using positron emission-tomography. In yet another embodiment, the CD24-specific targeting component is labeled with a paramagnetic label and is detected in a patient using magnetic resonance imaging (MRI).

The present invention is illustrated, but not limited, by the following examples.

### Examples

### Example 1

### Processing Primary Glioma Tissue Samples and Cell Lines for CD24 RNA Expression

Adult and fetal human samples were obtained from patients who consented to tissue use under protocols approved by the University of Rochester-Strong Memorial Hospital Research Subjects Review Board.

Tissue from adult cerebral cortex, subcortical white matter, and hippocampus resected from intractable epilepsy was used as controls. Tumors were graded by the attending neuropathologist in accordance with World Health Organization (WHO) established guidelines.

Tumor specimens were divided into three portions, the first portion was used for culture, the second was frozen in liquid nitrogen for molecular analysis, and the third portion was fixed in 4% PFA for immunohistochemical and histological analysis to ensure the representativity of the tissue.

Tissue processing and magnetic isolation of A2B5 cells was performed as described previously (Nunes et al., “Identification and Isolation of Multipotential Neural Progenitor Cells from the Subcortical White Matter of the Adult Human Brain,” Nat Med 9(4):439-447 (2003), which is hereby incorporated by reference in its entirety). Following dissociation, cells were resuspended in serum-free media (SF) consisting of DMEM/F12/N1 media supplemented with bFGF (20 ng/nal; Sigma), EGF (20 ng/ml), PDGF-AA (20 ng/ml; Sigma), and plated in a cell suspension culture dish for overnight recovery. Magnetic separation of A2B5 positive and negative cells was performed 24-48 h after dissociation. Glioma cells were further cultured in SF media or DMEM/F12/N1 supplemented with 5% platelet-derived fetal-bovine serum (PD-FBS). The U87, U251, and U373 cell lines were obtained from the American Type Culture Collection (ATCC, Manassas, Va.). Cell lines were cultured at 37° C. with 5% CO2 in DMEM or EMEM media supplemented with 10% Fetal-bovine Serum (FBS).

### Example 2

### Analysis of CD24 RNA Expression by Microarray Analysis

Immediately after sorting, RNA was extracted and purified using RNeasy (Qiagen, Chatsworth, Calif.) according to manufacturers' specifications. Genomic DNA contamination was removed using an on-column DNase digestion step. Twenty nanograms of total RNA was amplified using ribo-SPIA based amplification (Nugen, Inc), labeled, fragmented and hybridized to HG-U133 Plus 2.0 GeneChip (Affymetrix).

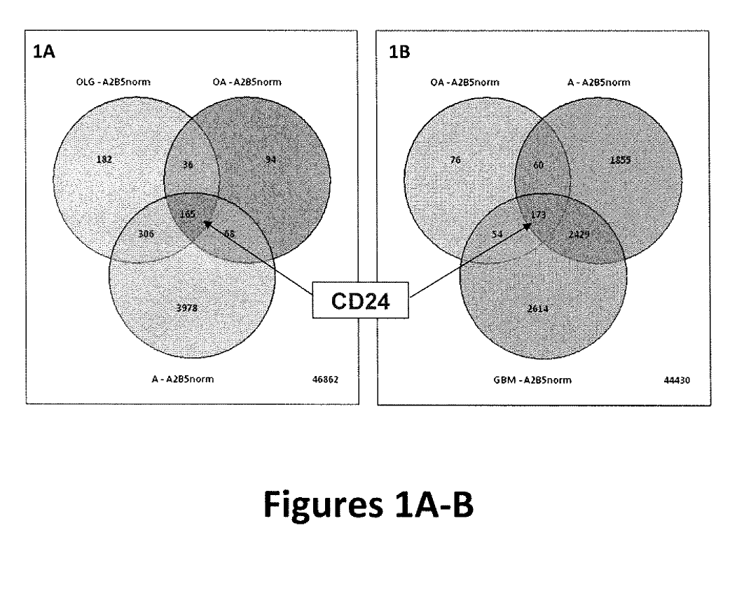

Microarray data was pre-processed using the RMA method (Irizarry et al., “Exploration, Normalization, and Summaries of High Density Oligonucleotide Array Probe Level Data,” Biostatistics 4(2):249-64 (2003), which is hereby incorporated by reference in its entirety) and downstream analysis performed using Bioconductor and R (Gentleman et al. “Bioconductor: Open Software Development for Computational Biology and Bioinformatics,” Genome Biol 5(10):R80 (2004), which is hereby incorporated by reference in its entirety. The gene expression profiles of A2B5-sorted glioma progenitors isolated from oligodendroglioma (n=4), oligoastrocytoma (n=4), astrocytoma (n=2), and glioblastoma multiforme (n=3) was analyzed. The expression profiles of glioma progenitors were compared directly to the expression profiles of normal adult human glial progenitor isolated from either adult sub-cortical white matter or cortex (n=4, each). These profiles were further filtered to remove transcripts non-specifically expressed in normal tissues. Genes having significantly enriched expression in unsorted white matter or cortical dissociates, or from human microglial cells purified from white matter on the basis of CD11b-immunoreactivity were removed from the analyses. Genes were defined as being significantly over expressed by A2B5⁺ tumor progenitor cells if the level of expression was 3-fold greater in tumor progenitor cells relative to normal progenitor cells. Significance was determined using a moderated t-test statistic with 5% false discovery rate cut-off (q<0.05, linear modeling empirical Bayes test statistic). Using this method, approximately 800 uniquely annotated genes were identified as enriched in tumor progenitors. FIGS. 1A-B are Venn diagrams illustrating the overlap in regulated mRNA transcripts in A2B5-isolated glioma tumor progenitor cells derived from the oligodendroglioma, oligoastrocytoma, astrocytoma, and glioblastoma multiforme tissue samples.

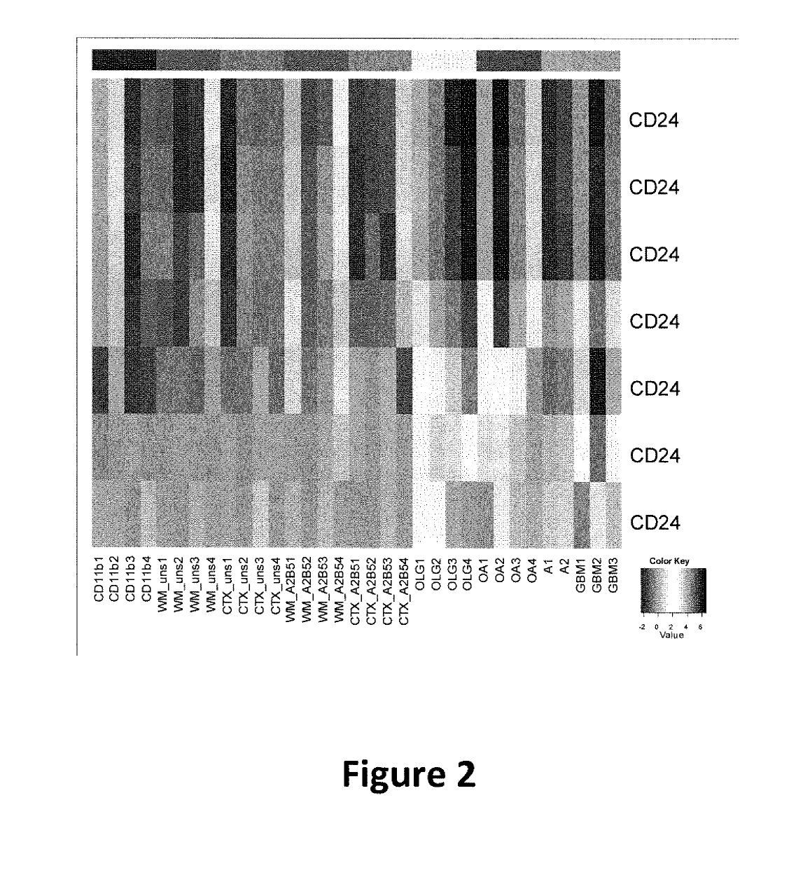

Among significant transcripts, the mRNA for CD24 was the most highly expressed cell-surface protein. Several probe sets annotated for CD24 detected a high level of expression in tumor progenitors (FIG. 2). The most significant of which was a 20.6-fold higher level of expression in A2B5⁺ tumor progenitors (q=4.63×10⁻⁶). CD24 mRNA was significantly over-expressed in tumor progenitors relative to normal progenitors in individual tumor sub-types. In summary, the array data identified CD24 mRNA as a tumor specific transcript that is significantly over expressed in A2B5+-tumor progenitor cells across glioma phenotypes and not highly expressed in normal human brain parenchyma.

### Example 3

### Validation of CD24 RNA Overexpression by Real-Time Polymerase Chain Reaction

CD24 mRNA expression was monitored by quantitative PCR (qPCR) using a TaqMan Gene expression assay for CD24 (Assays-on-Demand Hs00273561_s1*, Applied Biosystems, Foster City, Calif.). RNA was amplified using NuGen whole transcriptome amplification kit. Amplified cDNA was loaded in duplicate into Taqman low density arrays. Relative expression of each transcript was quantified using the ^{ΔΔ}Ct analysis and human GAPDH as a normalization control as described by the manufacturer (ABI, Foster City, Calif.). The mean, standard error, and significance testing of the individual samples were calculated on ^{ΔΔ}Ct values prior to anti-log transformation for data presentation. Significance was determined by comparing expression in tumor and normal sorted progenitors using and t test (p<0.005).

CD24 qPCR revealed an 18.76-fold increase in CD24 mRNA expression in A2B5-sorted tumor cells compared to their normal A2B5-sorted glial progenitor counterparts (5.39-65.29 fold higher, 95% confidence limit). The fold change per tumor phenotype was as following: 40.5-fold (p=0.002) in oligo-astrocytomas, 34.8-old (p=0.042) in astrocytomas, 19.1-fold (p=0.025) in oligodendrogliomas, and 14.1-fold (p=0.022) in glioblastomas. CD24 mRNA expression was not significantly regulated in A2B5-positive sorted tumor cells compared to A2B5-negative tumor cells.

### Example 4

### Validation of CD24 Expression by Immunohistochemistry in Tissue Sections

Human samples resected from fetal/adult brain and gliomas were immersed in paraformaldehyde (4%) in phosphate buffer (PB) for 1 to 2 hours at 4° C. according to the size of the sample, then gradually cryoprotected in sucrose (6% and 30%) in PB for 24 h at 4° C., embedded with Tissue-Tek OCT and frozen. Serial 14 μm sections were cut with a cryostat, dried at room temperature, and processed for immunohistochemical and histological analysis.

For each human sample, sections at different levels were stained with hematoxylin and eosin for assessment of histological parameters. For immunohistochemical labeling, sections were rehydrated in PBS and permeabilized for 15 minutes with PBS containing 0.1% saponin and 1% normal goat/donkey serum (NGS/NDS). Sections were washed three times in PBS and then incubated for 1 hour with PBS containing 0.05% saponin and 10% NGS/NDS. The primary antibodies were diluted in PBS containing 0.01% saponin and 5% NGS/NDS and incubated overnight. After three washes with PBS, sections were further incubated with a solution of secondary antibodies (in PBS containing 0.01% saponin and 5% NGS/NDS) using AlexaFluor488 and 594-labeled secondary antibodies ( 1/500- 1/1000, Molecular Probes). Sections were counter stained with 4′-6-diamidino-2-phenylindole (DAPI; Invitrogen). The following antibodies were used : CD24 (mouse IgG2a, clone ML5, BD Biosciences) (mouse IgG2a, Chemicon), Olig2 (Goat IgG, R&D), Sox2 (Goat IgG, R&D), Nestin (Rabbit IgG, Chemicon), GFAP (Rabbit IgG, Chemicon), PDGFRa (Rabbit IgG, Santa-Cruz Biotechnology), ClassIII-β-tubulin (Rabbit IgG, R&D). Immunofluorescence measurements were made using an epifluorescence and a confocal microscope (Olympus BX51) using ×40 and ×60 objectives.

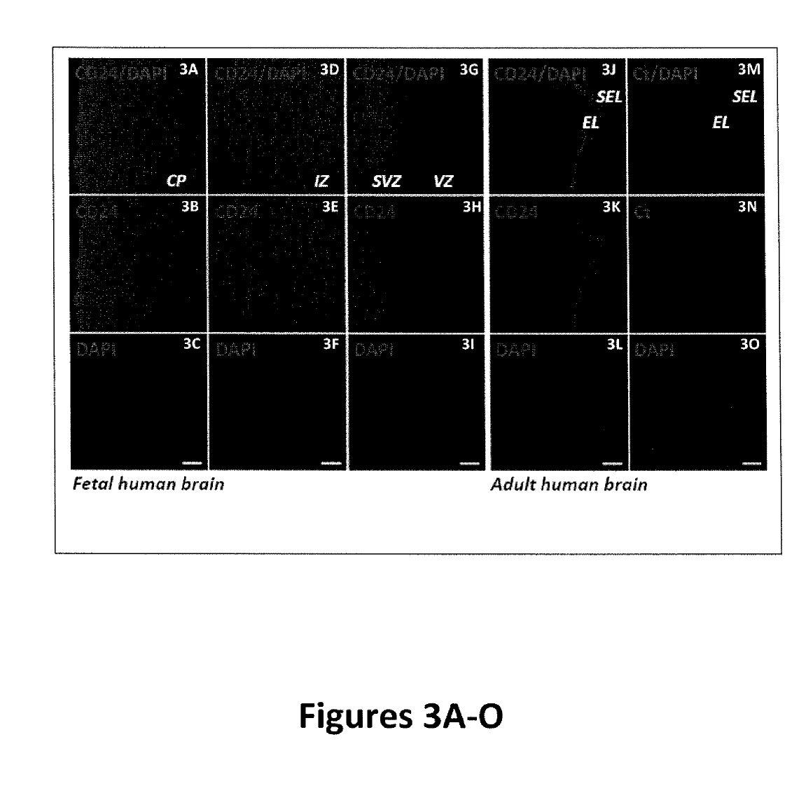

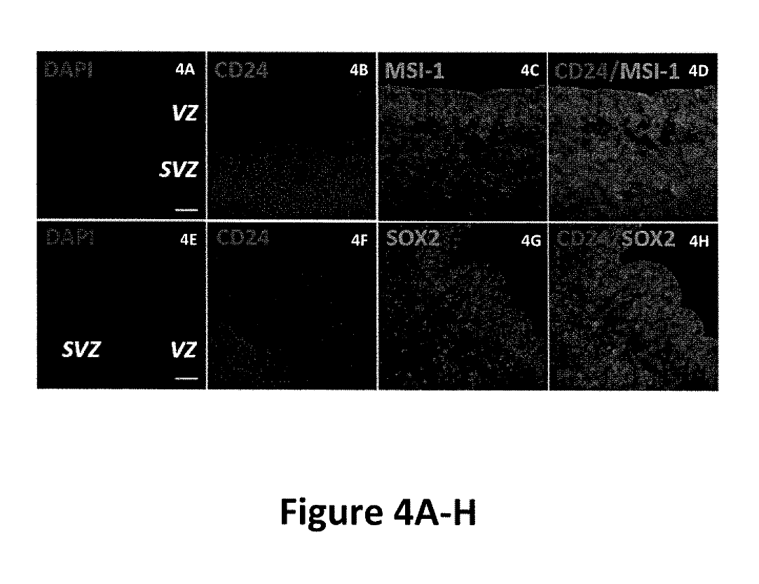

The expression of CD24 in low and high grade glioma samples of different phenotypes (n=21), adult human brain (n=6) and fetal human brain (n=3) were examined. As demonstrated in FIGS. 3A-1, a diffuse and intense pattern of immunoreactivity was observed in fetal human brain, from the cortical plate (CP) (FIGS. 3A-C), through the intermediate zone (IZ) (FIGS. 3D-F), to the sub-ventricular (SVZ) (FIGS. 3G-I). In contrast, a low degree of expression was observed in the ventricular zone (VZ) (FIGS. 3G-H and 4B and 4F), a region strongly immunoreactive for the stem/progenitor markers Sox2 and Musashi-1, as shown in FIGS. 4C and 4G. As shown in FIGS. 3J-L, a membranous pattern of CD24-immunoreactivity was restricted to the ependymal layer in normal adult hippocampus resected from pharmacoresistant epileptic patients. In addition, a diffuse expression of CD24 was observed into the sub-ependymal layer (SEL) (FIGS. 3J-K) as well as into the sub-granular zone and the granule cell layer of the gyrus dentatus.

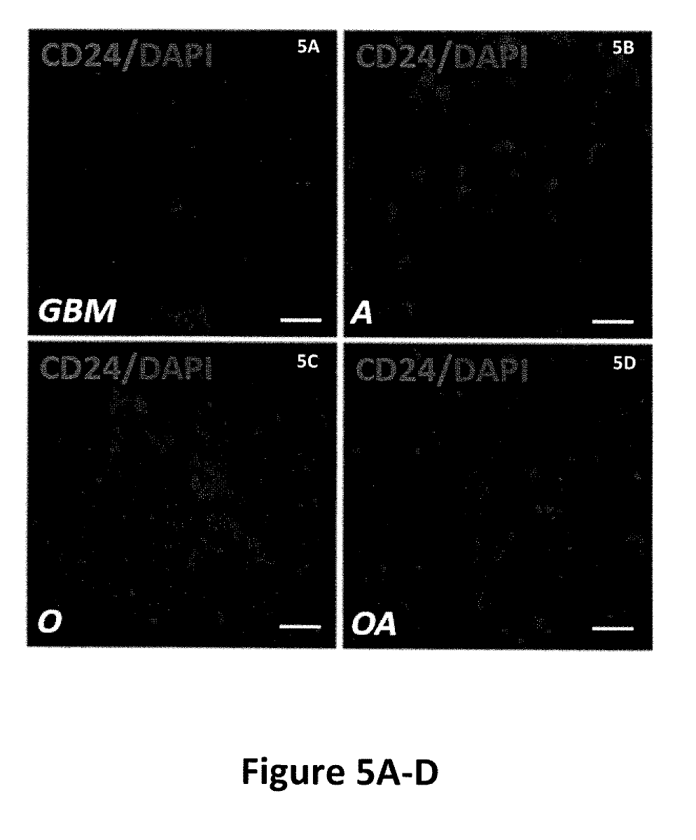

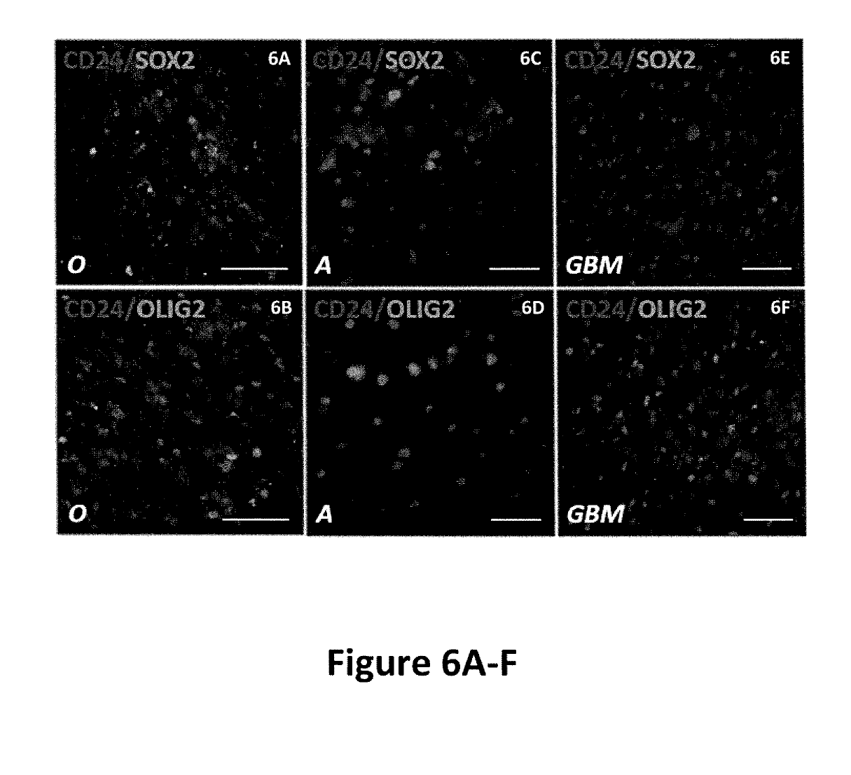

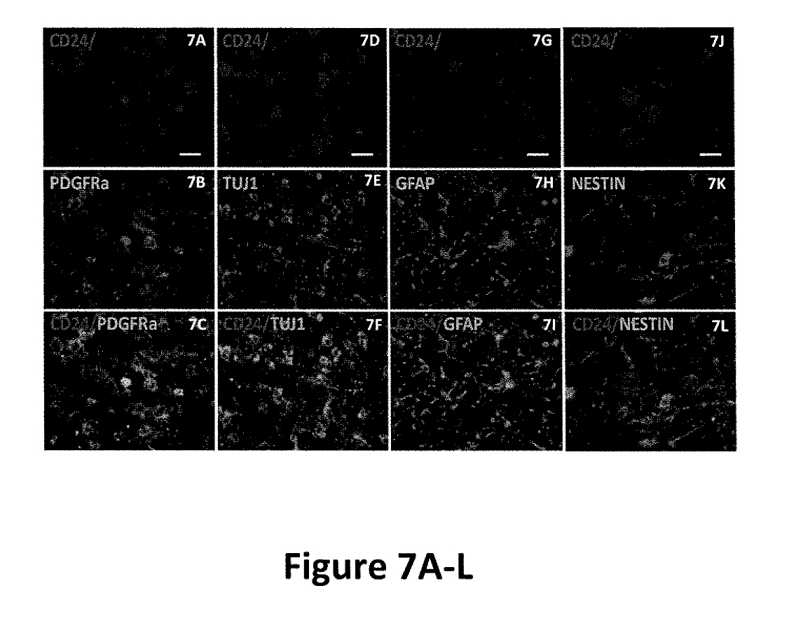

As shown in FIGS. 5 and 6, CD24 expression was observed in all tumor types and grades examined with variable degree of intensity and frequency in glioma samples. Two patterns were observed, a surface membrane and/or cytoplasmic pattern of labeling on subsets of cells allowing a clear morphological identification of immunoreactive cells and a diffuse pattern observed regionally excluding numerical quantification. A clear surface and/or cytoplasmic pattern was only observed for a subset of samples on which double labeling were performed. Double-labeling analysis revealed that the majority of CD24-immunoreactive cells co-express the progenitor markers Olig2 (88.24±5.88% of CD24+cells) (FIGS. 6B, D, and F) and Sox2 (84.81±11.75% of CD24+ cells) (FIGS. 6A, C, and E) in all tumors examined. Numerous CD24-immunoreactive cells were found to co-express the neuronal marker ClassIII-β Tubulin (Tuj1) (FIGS. 7D-F). A significant degree of co-labeling was demonstrated with the oligodendrocytic progenitor marker PDGFRα in oligodendroglial tumors (FIGS. 7A-C). None or very few cells co-expressed CD24 and the astrocytic marker GFAP (FIGS. 7G-I) or the stem cell marker Nestin (FIGS. 7J-L).

### Example 5

### Quantitative Flow Cytometry Analysis of CD24 Expression of Human Glioma—Derived Cell Lines

Cells of the U87, U373, U251, SID-227 gliomablastoma cell lines and the SID 238 gliosarcoma cell line (50000 to 100000 cells) were resuspended in 50 μl of flow cytometry (FC) buffer (PBS with 2 mM EDTA and 0.5% BSA), plated in 96 well plate and incubated for 20 minutes on ice with the following antibodies: CD133 (mouse IgG1; clone AC141-PE; Myltenyi Biotech), CD24 (clone ML5, mouse IgG2a; FITC-conjugated or unconjugated; BD Biosciences), and A2B5 (mouse IgM; Chemicon). Cells were washed in FC buffer and incubated with secondary fluorescent-conjugated antibody (goat anti-mouse IgM-APC conjugated or goat anti-mouse IgG-A488 conjugated) for 20 minutes at room temperature. Cells were washed once and resuspended in FC buffer supplemented with 4′,6-diamidino-2-phenylindole (DAPI, Invitrogen) to a final concentration of 80 ng/ml before analysis. Control conditions included unlabeled cells and cells labeled with appropriate isotypes control or secondary antibodies alone. Cells were analyzed on a FACS ARIA flow cytometer (BD Biosciences) using the FACS DIVA software and/or FlowJo.

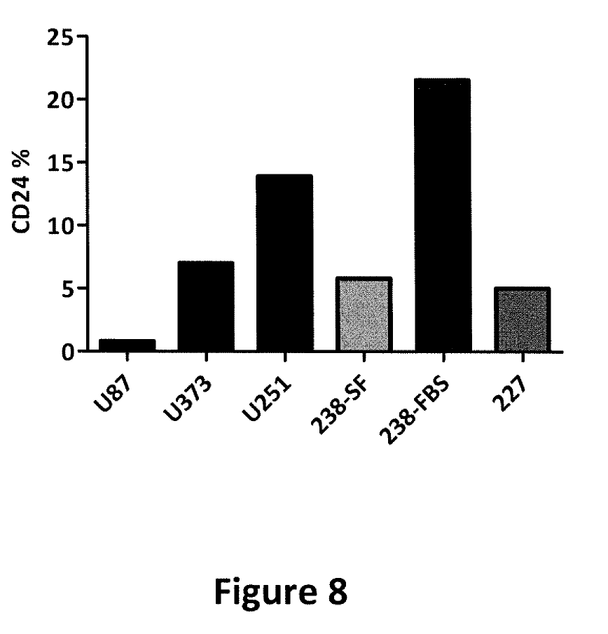

Several glioma cell lines demonstrated a significant degree of CD24-immunoreactivity, with the exception of the U87 cell line (See FIG. 8). A higher degree of expression was observed for the glioblastoma/gliosarcoma cell line SID238 maintained in culture media supplemented with 5% FBS compared to cells maintained in serum-free (SF) media supplemented with growth factors.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating a primary brain tumor in a subject, said method comprising:
administering to the subject a CD24-specific targeting component under conditions effective to treat the primary brain tumor in the subject.

2. The method of claim 1 further comprising:
selecting a subject at risk of having primary brain tumors, wherein the CD24-specific targeting component is administered to the selected subject.

3. The method of claim 1, wherein the CD24-specific-targeting component is linked to a chemotherapeutic or a radioisotope.

4. The method of claim 3, wherein the CD24-specific-targeting component is linked to a chemotherapeutic.

5. The method of claim 4, wherein the chemotherapeutic is diphtheria, ricin, or cholera toxin.

6. The method of claim 3, wherein the CD24-specific targeting agent is linked to a radioisotope.

7. The method of claim 1, wherein the primary brain tumor is selected from the group consisting of glioma, mixed oligo-astrocytoma, glioblastoma, anaplastic astrocytoma, oligodendroglioma, neurocytoma, primitive neuroectodermal tumor, dysplastic neuroepithelial tumor, and ganglioglioma.

8. The method of claim 1, wherein said administering is selected from the group consisting of systemic, intravascular, intraventricular, and intraparenchymal administration to the brain, brain stem, or spinal cord.

9. The method of claim 1, wherein the CD24-specific targeting component is an antibody.

10. The method of claim 1, wherein said administering decreases or abolishes tumor bulk.

11. The method of claim 1, wherein said administering diminishes tumor invasion and delays disease progression.

12. A method of preventing the migratory spread of a primary brain tumor in a subject, said method comprising:
administering to the subject a CD24-specific targeting component under conditions effective to prevent the migratory spread of a primary brain tumor within the subject.

13. The method of claim 12 further comprising:
selecting a subject at risk for migratory spread of tumors within the brain, wherein the CD24-specific targeting component is administered to the selected subject.

14. The method of claim 12, wherein the CD24-specific targeting component is linked to a chemotherapeutic or a radioisotope.

15. The method of claim 14, wherein a chemotherapeutic is linked to the CD24-specific targeting component.

16. The method of claim 15, wherein the chemotherapeutic is diphtheria, ricin, or cholera toxin.

17. The method of claim 14, wherein a radioisotope is linked to the CD24-specific targeting component.

18. The method of claim 12, wherein said administering is selected from the group consisting of systemic, intravascular, intraventricular, and intraparenchymal administration to the brain, brain stem, or spinal cord.

19. The method of claim 12, wherein the CD24-specific targeting component is an antibody.

20. A method of diagnosing the existence of a brain tumor in a subject, said method comprising:
providing a patient sample and
analyzing the patient sample for a presence of a CD24 antigen or a CD24-encoding nucleic acid molecule, thereby indicating the presence of a brain tumor in the subject.

21. The method of claim 20, wherein said analyzing is carried out with a CD24-specific antibody which binds to CD24 in an immunonological reaction.

22. The method of claim 20, wherein said analyzing is carried out with a CD24-encoding nucleic acid or its complement which binds to its complement in a hybridization reaction.

23. The method of claim 22, wherein said analyzing is carried out with a CD24-encoding nucleic acid or its complement which binds to its complement in an amplified hybridization reaction.

24. The method of claim 22, wherein the sample is a tissue sample.

25. A method of diagnosing the existence or monitoring the status of a brain tumor in a subject, said method comprising:
providing a subject and
analyzing the subject for the presence or anatomical distribution of a brain tumor based on a presence of a CD24 antigen thereby diagnosing the existence or monitoring the status of a brain tumor in the subject.

26. The method of claim 25, wherein said analyzing comprises:
imaging the brain of the subject.

27. The method of claim 25, wherein said analyzing is carried out with a CD24-specific antibody which binds to CD24 in an immunologic reaction.
